SEQUENCE LISTING (18 sequences)

SEQ ID NO: 1 - protein, 273 aa, Borrelia burgdorferi
MKKYLLGIGLILALIACKQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 2 - protein, 212 aa, Borrelia burgdorferi
MKKNTLSAILMTLFLFISCNNSGKDGNTSANSADESVKGPNLTEISKKITESNAVVLAVKEVETLLASIDEVAKKAIGNLIAQNGLNAGANQNGSLLAGAYVISTLIAEKLDGLKNSEELKEKIEDAKKCNKAFTDKLKSSHAELGIANGAATDANAKAAILKTNGTKDKGAQELEKLFESVKNLSKAAQETLNNSVKELTSPVVAESPKKP

SEQ ID NO: 3 - protein, 171 aa, Borrelia burgdorferi
MNKKMKMFIVYAVFILIGACKIHTSYDEQSSGESKVKKIEFSKFTVKIKNKDKSGNWTDLGDLVVRKEENGIDTGLNAGGHSATFFSLEEEVVNNFVKVMTEGGSFKTSLYYGYKEEQSVINGIQNKEIITKIEKIDGTEYITFSGDKIKNSGDKVAEYAISLEELKKNLK

SEQ ID NO: 4 - protein, 227 aa, Borrelia burgdorferi
MNKKMFIICAVFALIISCKNYATSKDLEGAVQDLESSEQNVKKTEQEIKKQVEGFLEILETKDLNKLDTKEIEKRIQELKEKIEKLDSKKTSIETYSEYEEKLKQIKEKLKGKADLEDKLKGLEDSLKKKKEERKKALEDAKKKFEEFKGQVGSATGVTTGHRAGNQGSIGAQAWQCANSLGLGVSYSSSTGTDSNELANKVIDDSIKKIDEELKNTIENNGEVKKE

SEQ ID NO: 5 - protein, 357 aa, Borrelia burgdorferi
MNTKKISSAILLTTFFVFINCKSQVADKDDPTNKFYQSVIQLGNGFLDVFTSFGGLVAEAFGFKSDPKKSDVKTYFTTVAAKLEKTKTDLNSLPKEKSDISSTTGKPDSTGSVGTAVEGAIKEVSELLDKLVKAVKTAEGASSGTAAIGEVVDNAAAAKAADKDSVTGIAKGIKEIVEAAGGSKKLKAAAAKGENNKGAGKLFGKAGDAAHGDSEAASKAAGAVSAVSGEQILSAIVKAAAAGDQEGKKPGEAKNPIAAAIGEGDGDAEFNQDGMKKDDQIAAAIALRGMAKDGKFAVKNDEKGKAEGAIKGAAESAVRKVLGAITGLIGDAVSSGLRKVGDSVKAASKETPPALNK

SEQ ID NO: 6 - protein, 178 aa, Borrelia burgdorferi
MEKYLSYIKKDDLDAIQLKLQELLASLHIFYSNLRGIHWNIKDTNFFVIHKKTQKLYEYIEKIIDIVAERSRMLGYDSEFRYSEFMKKSFIKELDIESTSNFLPSMESIVCSLTEILKNIFGMRKLIDTAGDYGTANIMDDIMSDLEKHLWMHKALLENCDCFCHDENESKCCECDAK

SEQ ID NO: 7 - protein, 336 aa, Borrelia burgdorferi
MIINHNTSAINASRNNAINAANLSKTQEKPSSGYRINRASDDAAGMGVSGKINAQIRGLSQASRNTSKAINFIQTTEGNLNEVEKVLVRMKELAVQSGNGTYSDSDRGSIQIEIEQLTDEINRIADQAQYNQMHMLSNKSASQNVKTAEELGMQPAKINTPASLSGSQASWTLRVHVGANQDEAIAVNIYSANVANLFAGEGAQAAQAAPVQEGAQEEGAQQPTPATAPSQGGVNSPVNVTTTVDANTSLAKIENAIRMISDQRANLGAFQNRLESIKNSTEYAIENLKASYAQIKDATMTDEVVAATTKSILTQSAMAMIAQANQVPQYVLSLLR

SEQ ID NO: 8 - protein, 672 aa, Borrelia burgdorferi
MKELDKEKLRDFVNMDLEFVNYKGPYDSTNTYEQIVGIGEFLARPLINSNSNSIYYGKYFINRFIDDQDKKASVDVFSIGSRSQLDSILNLRRILTGYLIKSFDYERSSAELIAKVITIHNAVYRGDLNYYKEVYIEAALKSLTKENAGLSRVYSQWAGKTQIFIPLKKNILSGKVESDIDIDSLVTDKVVAALLSENEAGVNFARDITDIQGETHKADQDKIDIELDNVHKSDSNITETIENLRDQLEKATDEEHRKEIESQVDAKKKQKEELDKKAIDLDKAQQKLDSSEDNLDIQRDTVREKIQEDIDEINKEKNLPKPGDVSSPKVDKQLQIKESLEDLQEQLKETSDENQKREIEKQIEIKKSDEELLKSKDPKALDLNGDLNSKVSSKEKIKGKEGEIVKEESKASLADLNNDENLMRPEDQKLSEDKKLDSKKNLKPVSEIERVNEISKSNNNEISESSPLYKPSYSDMDSKEGIDNKDVNLQETKSQTKSQPTSLNQDLTTMSIDSSNPVFLEVIDPITNLGTLQLIDLNTGVRLKESTQQGIQRYGIYEREKDLVVIKMDSGKAKLQILNKLENLKVISESNFEINKNSSLYVDSKMILVVVKDSGNVWRLAKFSPKNLNEFILSENKILPFTSFSVRKNFIYLQDEFKSLITLDLNTLKKVK

SEQ ID NO: 9 - protein, 339 aa, Borrelia burgdorferi
MNKILLLILLESIVFLSCSGKGSLGSEIPKVSLIIDGTFDDKSFNESALNGVKKVKEEFKIELVLKESSSNSYLSDLEGLKDAGSDLIWLIGYRFSDVAKVAALQNPDMKYAIIDPIYSNDPIPANLVGMTFRAQEGAFLTGYIAARLSKTGKIGFLGGIEGEIVDAFRYGYEAGAKYANKDIKIFTQYIGSFADLEAGRSVATRMYSDEIDIIHHAAGLGGIGAIEVAKELGSGHYIIGVDEDQAYLAPDNVITSTTKDVGRALNIFTSNHLKTNTFEGGKLINYGLKEGVVGFVRNPKMISFELEKEIDNLSSKIINKEIIVPSNKESYEKFLKEFI

SEQ ID NO: 10 - protein, 21 aa, Borrelia burgdorferi
KVTSKDKSSTEEKFNEKGEVS

SEQ ID NO: 11 - protein, 20 aa, Borrelia burgdorferi
NSGKDGNTSANSADESVKGP

SEQ ID NO: 12 - protein, 22 aa, Borrelia burgdorferi
FILIGACKIHTSYDEQSSGESK

SEQ ID NO: 13 - protein, 20 aa, Borrelia burgdorferi
VQDLESSEQNVKKTEQEIKK

SEQ ID NO: 14 - protein, 22 aa, Borrelia burgdorferi
FFVFINCKSQVADKDDPTNKFY

SEQ ID NO: 15 - protein, 20 aa, Borrelia burgdorferi
DKSFNESALNGVKKVKEEFK

SEQ ID NO: 16 - protein, 22 aa, Borrelia burgdorferi
SYIKKDDLDAIQLKLQELLASL

SEQ ID NO: 17 - protein, 21 aa, Borrelia burgdorferi
LSKTQEKLSSGYRINRASDDA

SEQ ID NO: 18 - protein, 23 aa, Borrelia burgdorferi
YKGPYDSTNTYEQIVGIGEFLAR

(19) United States
(12) Patent Application Publication
(10) Pub. No.: US 2026/0014239 A1
(43) Pub. Date: Jan. 15, 2026

(54) METHODS AND COMPOSITIONS FOR THE DIAGNOSIS, PROPHYLAXIS AND TREATMENT OF LYME DISEASE

(71) Applicant: Lankenau Institute for Medical Research, Wynnewood, PA (US)

(72) Inventor: Sunil Thomas, Philadelphia, PA (US)

(73) Assignee: Lankenau Institute for Medical Research, Wynnewood, PA (US)

(21) Appl. No.: 19/274,751

(22) Filed: Jul. 21, 2025

Related U.S. Application Data
(63) Continuation of application No. 17/534,970, filed on Nov. 24, 2021, now Pat. No. 12,364,744.
(60) Provisional application No. 63/118,101, filed on Nov. 25, 2020.

(51) Int. Cl. A61K 39/02; A61K 39/00; A61P 31/04; G01N 33/68

(52) U.S. Cl. CPC A61K 39/0225; A61P 31/04; G01N 33/6893; A61K 2039/521; A61K 2039/53; G01N 2333/20; G01N 2800/26

(57) ABSTRACT

Reagents that include certain identified peptides derived from B. burgdorferi proteins, or nucleic acid sequences encoding same are useful in the diagnosis of Lyme borreliosis (Lyme Disease) in infected subject are disclosed herein. Such reagents can also include nucleic acid sequences encoding the peptides. Pharmaceutical compositions useful for the treatment or prophylaxis of B. burgdorferi infection include certain peptides derived from B. burgdorferi proteins, or nucleic acid sequences encoding same. Methods of treating or preventing Lyme borreliosis (Lyme Disease) include administering effective amount of such pharmaceutical compositions. An antibody independent prophylactic pharmaceutical composition comprises a sonicate of B. burgdorferi bacterium; and methods of treating or preventing Lyme borreliosis include administering effective amounts of that sonicate composition are also disclosed herein.

### CROSS-REFERENCE TO RELATED APPLICATIONS

This is a continuation application of U.S. patent application Ser. No. 17/534,970, filed on Nov. 24, 2021, which claims priority to U.S. Provisional application 63/118,101filed Nov. 25, 2020, the entire contents of each being incorporated herein by reference as though set forth in full.

### INCORPORATION-BY-REFERENCE OF MATERIAL SUBMITTED IN ELECTRONIC FORM

The contents of the electronic sequence listing (MLH106USC1.xml; Size: 19,458 bytes; and Date of Creation: Jul. 14, 2025) is herein incorporated by reference in its entirety.

### FIELD OF THE INVENTION

This invention relates to reagents that include certain identified peptides derived from B. burgdorferi proteins, or nucleic acid sequences encoding same that are useful in the diagnosis, treatment, and prevention of Lyme borreliosis (Lyme Disease) in an infected subject.

### BACKGROUND OF THE INVENTION

Ticks are responsible for transmission of Lyme disease, ehrlichiosis, rickettsiosis, anaplasmosis, etc. Most of the diseases transmitted by ticks are neglected diseases as they affect only hundreds of people in the rural areas. However, such diseases have the potential to cause widespread infection under suitable conditions and are often misdiagnosed leading to fatalities. Lyme disease has become recognized as the most prevalent arthropod-borne infection in the United States (Centers for Disease Control, 1996). The incidence of Lyme disease is increasing over the years. There are no commercial vaccines available that effectively provide protection against Borrelia burgdorferi, the bacteria responsible for Lyme disease.

The genus Borrelia is a member of the family Spirochaetacaea (spirochetes). The incidence of Lyme disease in the United States has been increasing since national surveillance with the use of a standardized case definition was instituted in 1991. There is no way of knowing exactly how many people get Lyme disease. A recently released estimate based on insurance records suggests that each year approximately 76,000 Americans are diagnosed and treated for Lyme disease. This number is likely an over-estimate of actual infections because patients are sometimes treated presumptively in medical practice. Regardless, this number indicates a large burden on the health care system and the need for more effective prevention measures.

The great majority of cases occur in north-eastern United States, with additional foci in northern midwestern states (Wisconsin and Minnesota). Lyme disease also occurs in the Pacific coastal regions of Oregon and northern California. Although the geographic range of Lyme disease remains limited, it has been expanding, primarily due to changes in climate. The incidence of Lyme disease is highest among children 5 to 14 years of age and middle-aged adults (40 to 50 years of age), and it is slightly more common among males than among females.

The most common sign of Lyme disease is erythema migrans. Erythema migrans usually begins as a small erythematous papule or macule that appears at the site of the tick bite 1 to 2 weeks later and subsequently enlarges. The skin lesion is frequently accompanied by influenza-like symptoms, such as malaise and fatigue, headache, arthralgias, myalgias, fever, or regional lymphadenopathy, and these symptoms may be the presenting manifestation of the illness. The spread of B. burgdorferi within the nervous system has been demonstrated in nonhuman primates. In up to 5 percent of untreated patients, B. burgdorferi may cause chronic neuroborreliosis, sometimes after long periods of latent infection.

Clearly, effective methods for diagnosing, treating, and preventing Lyme Disease are needed.

### SUMMARY OF THE INVENTION

In one aspect, a reagent useful for the diagnosis of Lyme borreliosis in a subject comprises a peptide of SEQ ID NO: 10, 11, 12, 13, 14, 15, 16, 17 or 18. In another aspect, the reagent comprises a peptide having at least 90% identity to a peptide of any of SEQ ID NO: 10 through 18. In another aspect, the reagent comprises a peptide variant differing in one or more conservative amino acids to a peptide of any one of SEQ ID NO: 10 through 18. In another embodiment, the reagent comprises a peptide variant differing in one or more naturally occurring amino acids from a peptide of SEQ ID NO: 10 through 18, based upon the strain of B. burgdorferi from which the peptide sequence is derived.

In another aspect, a reagent comprises a mixture of multiple of the peptides identified herein.

In another aspect, a reagent useful for the diagnosis of Lyme borreliosis in a subject comprises a nucleic acid sequence encoding any of the peptides identified herein.

In another aspect, a reagent comprises a mixture of the nucleic acid sequences encoding multiple peptides identified herein.

In one aspect, a method for diagnosing Lyme borreliosis includes contacting a biological sample from a subject with one or more of the reagents described herein.

In yet another aspect, a pharmaceutical composition for the treatment, inhibition, or prophylaxis of Lyme borreliosis comprises a peptide of SEQ ID NO: 10, 11, 12, 13, 14, 15, 16, 17 or 18 and a pharmaceutically acceptable carrier, excipient, or adjuvant. In another aspect, the composition comprises a peptide having at least 90% identity to a peptide of any of SEQ ID NO: 10 through 18 and a pharmaceutically acceptable carrier, excipient, or adjuvant. In another aspect, the composition comprises a peptide variant differing in one or more conservative amino acids to a peptide of any one of SEQ ID NO: 10 through 18 and a pharmaceutically acceptable carrier, excipient, or adjuvant. In another embodiment, the composition comprises a peptide variant differing in one or more naturally occurring amino acids from a peptide of SEQ ID NO: 10 through 18, based upon the strain of B. burgdorferi from which the peptide sequence is derived and a pharmaceutically acceptable carrier, excipient, or adjuvant. In still another embodiment, this composition contains two or more of these peptides in admixture and a pharmaceutically acceptable carrier, excipient, or adjuvant.

In another aspect, a pharmaceutical composition for the treatment, inhibition, or prophylaxis of Lyme borreliosis comprises a nucleic acid sequence encoding any of the peptides identified herein and a pharmaceutically acceptable carrier, excipient, or adjuvant. In still another embodiment, this composition further contains a single nucleic acid sequence encoding multiple different peptides described herein, or a mixture of different nucleic acid sequences, each sequence encoding a different peptide described herein.

Yet another aspect provides a method of treating, preventing, inhibiting, or retarding infection by B. burgdorferi comprising administering to a subject in need thereof an effective amount of a pharmaceutical composition as described herein.

In another aspect, a pharmaceutical composition for the treatment, retarding or prophylaxis of Lyme borreliosis comprises a sonicate of Borrelia burgdorferi.

In yet another aspect, a method of treating, preventing, or retarding infection comprises administering to a subject in need thereof, an effective amount of a pharmaceutical composition comprising a sonicate of Borrelia burgdorferi, wherein the sonicate elicits an antibody-independent protective immune response.

Still other aspects and advantages of these compositions and methods are described further in the following detailed description of the preferred embodiments thereof.

### BRIEF DESCRIPTION OF THE DRAWINGS

FIG. 1A-1D are hydrophobic plots of the antigens of Borrelia, OspA, OspC, P100 and VISE, respectively. The epitopes are selected based on hydrophilicity.

FIG. 2 is a graph showing the detection of Borrelia specific antibodies in blood samples (positive: (● known patients) (negative: ▪ healthy controls).

FIG. 3 is a graph showing that the antigenic peptides of Borrelia reacted with peptide-specific antibody before (dark bar) and after (light bar) challenge with the pathogen.

FIG. 4 is a graph showing bacterial load. C3H/HeN mice were immunized at day 0 and 14 and were infected at 4 weeks post-priming with 10⁵ Borrelia cells per mouse.

Two weeks post-infection mice were euthanized, and tissues were collected to evaluate bacterial burden. The tissues are identified on the X axis and the various peptides and controls are identified below each bar.

### DETAILED DESCRIPTION

In response to the continuing need in the art that exists for new and effective tools and methods for the diagnosis, prophylaxis, and treatment of Lyme Disease, the inventors have identified and synthesized certain peptides corresponding to the epitopes of the antigenic proteins of Borrelia. These antigenic peptides are useful in diagnostics application to detect Borrelia-specific antibodies in Lyme disease patient biological samples. In another embodiment, these structure-based peptides induce a protective immune response (e.g., antibodies that are protective against Borrelia in animal models). These peptides thus are useful in Lyme disease vaccine compositions. Additionally, the inventors disclose a sonicated bacteria vaccine that does not induce antibody but demonstrates antibody-independent protection against Lyme disease.

In this specification, technical and scientific terms used herein have the same meaning as commonly understood by one of ordinary skill in the art to which this invention belongs and by reference to published texts, which provide one skilled in the art with a general guide to many of the terms used in the present application. The definitions contained in this specification are provided for clarity in describing the components and compositions herein and are not intended to limit the claimed invention.

As used herein, “subject” refers to any species that can be infected by Borrelia burgdorferi. In certain embodiments the subject is a human. In certain embodiments, the subject is a non-human animal. In certain embodiments the subject is bovine, equine, porcine, canine or a feline.

An “effective immunizing amount,” as used herein, may vary depending upon the peptide(s), nucleic acid sequence(s), the Borrelia burgdorferi protein from which the peptide or nucleic acid sequence was derived, as well as depending upon the strain of Borrelia burgdorferi from which the peptides are derived, or which is used to generate the sonicate. In certain embodiment, the amount may be any amount sufficient to evoke a protective antibody-independent immune response. In another embodiments, the amount may be any amount sufficient to evoke an antibody response.

A “protective immune response” as used herein is an immune response in a subject that leads to protection against one or more indications of infection; and may span a range of effects from complete protection from any indication of B. burgdorferi infection to an immune response that permits reduction of symptoms of the underlying infection, after challenge. A protective immune response further includes elimination, reduction, or inhibition of one or more of the underlying cellular, physiological, or biochemical causes or mechanisms causing the symptoms. The pharmaceutical compositions described herein are intended to provide a protective immune response.

The term “percent identical” or “percent identity” refers to sequence identity between two amino acid sequences or two nucleic acid sequences. Identity can be determined by comparing a position in each sequence which may be aligned for purposes of comparison. When an equivalent position in the compared sequences is occupied by the same amino acid or nucleotide, then the amino acid or nucleic acid sequences are identical at that position. Expression as a percentage of identity refers to a function of the number of identical amino acids or nucleotides, or a derivative or variant thereof, at corresponding positions (e.g., as defined by an alignment) shared by the compared sequences. Various alignment algorithms and/or programs may be used to determine percent identity, non-limiting examples of which include FASTA, BLAST, or ENTREZ. FASTA and BLAST are available as a part of the GCG sequence analysis package (University of Wisconsin, Madison, WI), and can be used with, e.g., default settings. ENTREZ is available through the National Center for Biotechnology Information, National Library of Medicine, National Institutes of Health, Bethesda, MD.

As used herein, the term “biological sample” refers to, without limitation, material for testing obtain from a subject suspected of having Lyme Disease. These samples include blood, including serum, plasma, whole blood, and peripheral blood, saliva, urine, vaginal or cervical secretions, amniotic fluid, placental fluid, cerebrospinal fluid, or serous fluids, mucosal secretions (e.g., buccal, vaginal, or rectal). Still other samples include a blood-derived or biopsy-derived biological sample of tissue or a cell lysate (i.e., a mixture derived from tissue, e.g., heart and joints) and/or cells.

By the term “immobilization surface” is meant any surface on which the peptide or nucleic acid in question can be bound, as opposed to use in solution. A variety of immobilization surfaces are useful for diagnostic testing, such as glass, silicon, metal plastic, PDMS, nitrocellulose, or hydrogel. The surface, depending upon the reagent and number of reagents and assay can be a planar microchannel, a microfluidics card, a plate, a slide, a chip, a bead, a microarray, a strip, or a self-assembled monolayer. See, e.g., Kim D and Herr, A, 2013 July, Biomicrofluidics, 7(4):041501 for lists of other surfaces. Methods of immobilizing the peptides or nucleic acids, covalently or non-covalently to such immobilization surfaces are known in the art. For example, the peptide or nucleic acid sequence is attached to a selected surface resulting in reduction or loss of mobility via physical adsorption through charge-charge interaction or hydrophobic interaction, covalent bonding, Streptavidin-Biotin interaction, or affinity coupling, among other techniques.

As used herein, the term “detectable label” means a reagent, moiety, or compound capable of providing a detectable signal, depending upon the assay format employed. A label may be associated with one or more peptides or one or more nucleic acid sequences. Alternatively, different labels may be used for each peptide or nucleic acid in the reagent or kit. Such labels are capable, alone or in concert with other compositions or compounds, of providing a detectable signal. In one embodiment, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength. Still other label systems that may be utilized in the described methods and reagents are detectable by other means, e.g., colored latex microparticles (Bangs Laboratories, Indiana) in which a dye is embedded may be used in place of enzymes to provide a visual signal indicative of the presence of the labeled ligand or construct in applicable assays. Still other labels include fluorescent compounds, fluorophores, radioactive compounds, or elements. In one embodiment, a fluorescent detectable fluorochrome, e.g., fluorescein isothiocyanate (FITC), phycoerythrin (PE), allophycocyanin (APC), coriphosphine-O (CPO) or tandem dyes, PE-cyanin-5 or-7 (PC5 or PC7)), PE-Texas Red (ECD), PE-cyanin-5.5, rhodamine, PerCP, and Alexa dyes. Combinations of such labels, such as Texas Red and rhodamine, FITC+PE, FITC+PECy5 and PE +PECy7, among others may be used depending upon assay method. The selection and/or generation of suitable labels for use in labeling the peptide or nucleic acid reagents and/or any component of diagnostic kit is within the skill of the art, provided with this specification.

The terms “a” or “an” refers to one or more. For example, “an expression cassette” is understood to represent one or more such cassettes. As such, the terms “a” (or “an”), “one or more,” and “at least one” are used interchangeably herein.

As used herein, the term “about” means a variability of plus or minus 10% from the reference given, unless otherwise specified.

The words “comprise”, “comprises”, and “comprising” are to be interpreted inclusively rather than exclusively, i.e., to include other unspecified components or process steps. The words “consist”, “consisting”, and its variants, are to be interpreted exclusively, rather than inclusively, i.e., to exclude components or steps not specifically recited.

### B. burgdorferi Structure

Borrelia burgdorferi is a Gram-negative spiral bacterium bound by an inner cytoplasmic membrane and an outer membrane. The outer membrane lacks lipopolysaccharide and consists of a lipid bilayer that is composed of phospholipids and glycolipids. Cholesterol glycolipids in the outer membrane form lipid-raft-like microdomains that change in order and size in response to temperature, which is an important environmental cue for B. burgdorferi during transmission between the tick vector and the mammalian host. The B. burgdorferi outer membrane also contains surface lipoproteins, which can change depending on the environment. The outer surface proteins of B. burgdorferi includes OspA, OspB, OspC, OspD, OspE, OspF, DbpA, DbpB, CspA, VlsE, BptA that are lipid-modified outer surface proteins anchored to the outer leaflet of the outer membrane through their lipid moieties. P13, P66, BesC, BamA, Lmp1 and BB0405 are outer surface proteins that have one or more transmembrane domains that anchor them into the outer membrane.

B. burgdorferi bacteria live in the midgut of ticks. In ticks, B. burgdorferi predominantly express the outer-surface protein A (OspA) before the blood meal, when the bacteria is in the midgut. OspA mediates the attachment of B. burgdorferi to the tick midgut by binding the midgut receptor TROSPA (Tick Receptor for OspA). When infected ticks feed blood meal, the spirochetes multiply within the gut, express high levels of OspC, migrate to the tick's salivary glands, and infect the vertebrate host. OspC has been shown to bind a tick salivary protein, Salp15, in vitro and in vivo, indicating a possible role for OspC in transmission and/or survival early during host colonization. Thus, OspA is required for colonizing the vector; whereas, OspC is required for infecting the host. OspF is identified as a potential adhesin and binds to organs of the human host. B. burgdorferi is able to persist in patients for extended periods and establish chronic infection in host tissues. VIsE as an important virulence determinant of B. burgdorferi.

The periplasmic flagella of B. burgdorferi is responsible for the flat-wave morphology of the bacteria. The flagella are attached to each cell pole and wind around the cell cylinder in the periplasmic space between the peptidoglycan layer and the outer membrane. Flagellar motors are located at the cell poles and are situated next to the methyl-accepting chemotaxis proteins that direct movement of the bacteria towards chemoattractants (including nutrients) and away from repellants including organic solvents.

The genome of the bacterium Borrelia burgdorferi, contains a linear chromosome of 910.725 base pairs and at least 17 linear and circular plasmids. The chromosome contains 853 genes encoding a basic set of proteins for DNA replication, transcription, translation, solute transport and energy metabolism.

B. burgdorferi has a very limited metabolic capacity and is highly dependent on its tick vector and vertebrate host for most essential factors. B. burgdorferi lacks genes encoding proteins that have a role in the tricarboxylic acid cycle and oxidative phosphorylation, and relies exclusively on glycolysis for energy production. For this purpose, B. burgdorferi uses several host or vector-derived carbohydrates, including glucose, glycerol, maltose, N-acetylglucosamine, trehalose and chitobiose. The B. burgdorferi genome also lacks genes that are required for the synthesis of amino acids, lipids, nucleotides and cofactors. To obtain these factors, the B. burgdorferi genome encodes 16 distinct membrane transporters, many of which have broad substrate specificity. Owing to the inability of B. burgdorferi to synthesize fatty acids, its lipid composition reflects that of the host tissues; B. burgdorferi exchanges lipids with the plasma membrane of eukaryotic cells, either through direct contact or via outer membrane vesicles.

### Peptides and Nucleic Acid Sequences

Peptides useful in this invention were derived from B. burgdorferi outer surface proteins (Osp), i.e., specifically OspA, OspC, OspE, and OspF, VLSE, the neutrophil activating protein NapA, and proteins P41, P100 and the Laminin-binding Borrelia membrane protein A (BmpA) (see Example 1, Table 1 below). In one embodiment, the peptides are those identified as SEQ ID Nos: 10 to 18 (see Example 1, Table 2 below). In addition to the specified peptides

SEQ ID NO: 10
KVTSKDKS STEEKFNEKG EVS
SEQ ID NO: 11
NSGKDGNTSANSADESVKGP
SEQ ID NO: 12
FILIGACKIHTSYDEQSSGESK
SEQ ID NO: 13
VQDLESSEQNVKKTEQEIKK
SEQ ID NO: 14
FFVFINCKSQVADKDDPTNKFY
SEQ ID NO: 15
DKSFNESALNGVKKVKEEFK
SEQ ID NO: 16
SYIKKDDLDAIQLKLQELLASL
SEQ ID NO: 17
LSKTQEKLSSGYRINRASDDA
SEQ ID NO: 18
YKGPYDSTNTYEQIVGIGEFLAR,

other related peptides that may be useful in the reagents and compositions described herein.

One related peptide is a peptide having at least 90% identity to a specifically identified peptide. The phrase “at least 90% identity” is intended to include peptides having percent identities of 91, 92, 93, 94, 95, 96, 97, 99 and 99% as well as complete 100% identify with the specified peptide. In one embodiment, a peptide having at least 90% identity to a peptide of SEQ ID NO: 10 may be a usual peptide for diagnostic or therapeutic use. In another embodiment, a peptide having at least 90% identity to a peptide of SEQ ID NO: 11 may be a usual peptide for diagnostic or therapeutic use. In another embodiment, a peptide having at least 90% identity to a peptide of SEQ ID NO: 12 may be a usual peptide for diagnostic or therapeutic use. In another embodiment, a peptide having at least 90% identity to a peptide of SEQ ID NO: 13 may be a usual peptide for diagnostic or therapeutic use. In another embodiment, a peptide having at least 90% identity to a peptide of SEQ ID NO: 14 may be a usual peptide for diagnostic or therapeutic use. In another embodiment, a peptide having at least 90% identity to a peptide of SEQ ID NO: 15 may be a usual peptide for diagnostic or therapeutic use. In another embodiment, a peptide having at least 90% identity to a peptide of SEQ ID NO: 16 may be a usual peptide for diagnostic or therapeutic use. In another embodiment, a peptide having at least 90% identity to a peptide of SEQ ID NO: 17 may be a usual peptide for diagnostic or therapeutic use. In another embodiment, a peptide having at least 90% identity to a peptide of SEQ ID NO: 18 may be a usual peptide for diagnostic or therapeutic use.

Another related peptide is a peptide variant that differs from a specific peptide of SEQ ID NO: 10-18 by one or more conservative substitutions or replacements. For example, a conservative substitute is an amino acid replacement that changes a given amino acid to a different amino acid having similar biochemical properties, e.g., charge, hydrophobicity, and size). Such conservative amino acids include Met, Ile, Leu, and Val; or Phe with Trp; or Asp with Glu; or Lys and Arg; of His, Asp, Gln, Ser, Thr and Tyr. See, e.g., Betts M R and Russell R B, Amino Acid Properties and Consequences of Substitutions” in Bioinformatics for Geneticists, Eds. Barnes M R and Gray IC, 2003 John Wiley & Sons, Ltd.

Another related peptide is a peptide variant differing in one or more naturally occurring amino acids from a peptide of SEQ ID NO: 10-18, based upon the strain of B. burgdorferi from which the peptide sequence is derived.

Any nucleic acid sequence that encodes one or more of peptides SEQ ID NO: 10-18 or their related peptides as defined above is included in the compositions described herein. Because the genetic code is degenerate, one of skill in the art can construct a variety of differing nucleic acid sequences that can encode the same peptide. Thus, in embodiments of the compositions and methods described herein, it is understood that all nucleic acid sequences encoding the same peptides can be employed. Similarly, the encoding nucleic acid sequences can encode multiples of the same or multiple different peptides in a single sequence, if desired.

### Diagnostic Reagents and Methods

Thus, diagnostic reagents as disclosed herein can include an individual peptide or multiple peptides as defined herein. The multiple peptides can be present in a simple mixture or prepared as fusion proteins; or fused to carriers or immobilization surfaces or linked to detectable labels. The diagnostic compositions may contain multiple copies of at least 2, 3, 4, 5, 6, 7, 8, 9 or at least 10 different peptides from SEQ ID Nos 10-18 or related peptides. In other embodiments, optional amino acids (e.g.,-Gly-Ser-, or Pro-Ala or other equivalents) or chemical compound spacers may be included at the termini of the peptides for linking the peptides together or to a carrier or to an immobilization surface. The reagents may contain mixtures or individual peptides. Still other reagents can include peptides that contain fusions of two or more of SEQ ID Nos: 10-18. Still other suitable related peptides can be SEQ ID NO: 10-18 to which modifications are made on the N or C terminus. These peptides described herein may be produced synthetically or recombinantly. The reagent composition may take the form of one or more of the above-described peptides expressed as a synthetic peptide coupled to a carrier protein. Alternatively, a composition may contain multiple peptides, each expressed as a multiple antigenic peptide, optionally coupled to carrier protein. Alternatively, the selected peptides may be linked sequentially and expressed within a recombinantly produced protein.

By “mixture” is meant any combination of two or more different peptides (including, e.g., any of the variants or sequences that share at least 90% identity with the reference sequence or are natural variants or contain conservative replacements). In one embodiment, the reagent contains SEQ ID Nos.: 10 and 11, or 10 and 12, or 10 and 13, or 10 and 14, or 10 and 15, or 10 and 16, or 10 and 17, or 10 and 18, or 11 and 12, 11 and 13, or 11 and 14, or 11 and 15, or 11 and 16, or 11 and 17, or 11 and 18, or 12 and 13, or 12 and 14, or 12 and 15, or 12 and 16, or 12 and 17, or 12 and 18, or 13 and 14, or 13 and 15, or 13 and 16, or 13 and 17, or 13 and 18, or 14 and 15, or 14 and 16, or 14 and 17, or 14 and 18, or 15 and 16, or 15 and 17, or 15 and 18, or 16 and 17, or 16 and 18, or 17 and 18 (including, e.g., any of the variants or sequences that share at least 90% identity with the reference sequence, or are natural variants or contain conservative replacements). In another embodiment, the reagent contains SEQ ID Nos: 10, 11 and 12, or 10, 11 and 13, or 10, 11 and 14, or 10, 11 and 15, or 10, 11 and 16; or 10, 11 and 17, or 10, 11 and 18, or 10, 12, and 13, or 10, 12 and 14, or 10, 12 and 15, or 10, 12 and 16; or 10, 12 and 17, or 10, 12 and 18, or 10, 13, and 14, or 10, 13 and 15, or 10, 13 and 16; or 10, 13 and 17, or 10, 13 and 18, or 10, 14 and 15, or 10, 14 and 16; or 10, 14 and 17, or 10, 14 and 18 or 10, 15 and 16; or 10, 15 and 17, or 10, 15 and 18; or 10, 16 and 17, or 10, 16 and 18; or 10, 17 and 18; or 11, 12 and 13, or 11, 12 and 14, or 11, 12 and 15, or 11, 12 and 16; or 11, 12 and 17, or 11, 12 and 18, or 11, 13 and 14, or 11, 13 and 15, or 11, 13 and 16; or 11, 13 and 17, or 11, 13 and 18, or 11, 14 and 15, or 11, 14 and 16; or 11, 14 and 17, or 11, 14 and 18, or 11, 15 and 16; or 11, 15 and 17, or 11, 15 and 18, or 11, 16 and 17, or 11, 16, and 18; or 11, 17 and 18; or 12, 13 and 14, or 12, 13 and 15, or 12, 13 and 16; or 12, 13 and 17, or 12, 13 and 18, or 12, 14 and 15, or 12, 14 and 16; or 12, 14 and 17, or 12, 14 and 18, or 12, 15 and 16; or 12, 15 and 17, or 12, 15 and 18, or 12, 16 and 17, or 12, 16 and 18 or 12, 17 and 18; 13, 14 and 15, or 13, 14 and 16; or 13, 14 and 17, or 13, 14 and 18, or 14, 15, and 16, or 14, 15 and 17, or 14, 15 and 18; or 15, 16 and 17; or 15, 16 and 18; or 16, 17 and 18 (including, e.g., any of the variants or sequences that share at least 90% identity with the reference sequence, or are natural variants or contain conservative replacements).

In another mixture, the reagents include 4 peptides, including any combination of 4 peptides from SEQ ID No. 10-18 and any of the variants or sequences that share at least 90% identity with the reference sequence, or are natural variants or contain conservative replacements. In another mixture, the reagents include 5 peptides, including any combination of 5 peptides from SEQ ID No. 10-18 and any of the variants or sequences that share at least 90% identity with the reference sequence, or are natural variants or contain conservative replacements. In another mixture, the reagents include 6 peptides, including any combination of 6 peptides from SEQ ID No. 10-18 and any of the variants or sequences that share at least 90% identity with the reference sequence, or are natural variants or contain conservative replacements. In another mixture, the reagents include 7 peptides, including any combination of 7 peptides from SEQ ID No. 10-18 and any of the variants or sequences that share at least 90% identity with the reference sequence, or are natural variants or contain conservative replacements. In another mixture, the reagents include 8 peptides, including any combination of 8 peptides from SEQ ID No. 10-18 and any of the variants or sequences that share at least 90% identity with the reference sequence, or are natural variants or contain conservative replacements. In another mixture, the reagents include 9 peptides, including any combination of 9 peptides from SEQ ID No. 10-18 and any of the variants or sequences that share at least 90% identity with the reference sequence, or are natural variants or contain conservative replacements. In another mixture, the reagents include greater than 9 peptides, including any combination of >9 peptides from SEQ ID No. 10-18 and any of the variants or sequences that share at least 90% identity with the reference sequence, or are natural variants or contain conservative replacements.

Another form of diagnostic reagent depending upon the assay technique being used is a nucleic acid sequence that encodes one or more of peptides SEQ ID NO: 10-18 or their related peptides as defined above is included in the reagent compositions described herein. Such reagents can include nucleic acid sequences encoding multiple repetitions of a single peptide or multiple of the peptides in a fusion and may be produced by conventional synthetic or recombinant techniques. The multiple peptides may be selected from those mixtures described above.

In other embodiments of the reagents, one or more of the peptides or nucleic acid molecules is covalently or noncovalently joined to an immobilization surface, as defined above. Still other reagents include one or more of said peptides or nucleic acid molecules covalently or noncovalently joined to a labeling moiety capable alone or in combination with one or more additional molecules of generating a detectable signal. Examples of such labels and immobilization surfaces are described above.

Where the reagent is a nucleic acid sequence, such sequence can be labeled by suitable techniques or bound to immobilization surfaces by conventional binding techniques. Such nucleic acid sequences can be incorporated into plasmid or viral vectors, for recombinant expression by now common techniques known to one of skill in the art. Sec, textbooks such as Maniatis, T., et al (1982). Molecular cloning: a laboratory manual. Cold Spring Harbor, N.Y., Cold Spring Harbor Laboratory and others. In one embodiment, the DNA sequences can be employed as hybridization probes in hybridization assays. In other embodiment, the sequences can be employed as primers.

Diagnostic kits can include any of the above recited peptides or nucleic acid sequences, as wells immobilization surfaces, reagents for accomplishing the immobilization and other physical and chemical apparatus for performing the diagnostic assay of choice. In addition to the reagents above, alternatively, a diagnostic kit thus also contains miscellaneous reagents and apparatus for reading labels, e.g., certain substrates that interact with an enzymatic label to produce a color signal, etc., apparatus for taking blood samples, as well as appropriate vials and other diagnostic assay components.

The particular assay format used to measure the infection of a subject by B. burgdorferi may be selected from among a wide range of immunoassays to identify anti-B. burgdorferi antibodies, such as enzyme-linked immunoassays, sandwich immunoassays, homogenous assays, immunohistochemistry formats, or other conventional assay formats. One of skill in the art may readily select from any number of conventional immunoassay formats. Other assays include high pressure liquid chromatography (HPLC), immunohistochemistry, etc. In one embodiment, the diagnostic method is an Enzyme-Linked Immunosorbent Assay. Other suitable assay formats include detection protocols, including without limitation, PCR, Immuno-PCR and proximity ligation or proximity extension assay protocols, PEA, RCA, sequencing, and fluorescence hybridization protocols. There are a variety of assay formats known to the skilled artisan for using a ligand to detect a target molecule in a sample. (For example, see Harlow and Lane, Antibodies: A Laboratory Manual, Cold Spring Harbor Laboratory, 1988).

Thus, a method of diagnosing Lyme borreliosis in a subject, comprises contacting in vitro a biological sample from the subject with a peptide reagent or nucleic reagent such as described herein. The presence of an antibody that binds Borrelia is detected by the complex formed between the reagent and the antibody in the sample. Detection or measurement of the sample antibody to Borrelia may be obtained by use of a variety of apparatus or machines, such as computer-programmed instruments that can transform the detectable signals generated from the diagnostic reagents complexed with the antibody in the biological sample into numerical or graphical data useful in performing the diagnosis. Such instruments may be suitably programmed to permit the comparison of the measured antibody in the sample with the appropriate reference standard and generate a diagnostic report or graph that can indicate a diagnosis of infection, i.e., Lyme Disease.

In fact, as described in the examples, one assay format/method of diagnosis involved coating multiple of the peptides of SEQ ID NO: 10-18 on an ELISA plate; and incubating same with the blood samples of patients and controls. They were then probed with the secondary antibodies conjugated to horseradish peroxidase, for use in generating a detectable signal if antibodies in the sample bound to the peptides on the plate, indicating the presence of antibodies to B. burgdorferi. The data in the examples demonstrated that the tested peptides are useful in the diagnosis of Borrelia in patients. The peptides are also potential vaccine candidates and could be used to protect against Borrelia.

Other assay formats may be employed using the reagents described herein to detect Lyme Disease in biological samples.

### Pharmaceutical Compositions and Methods

The peptides described above and nucleic acid sequences encoding them are also useful in pharmaceutical composition and methods for treating, retarding or prevention Lyme Disease in subjects. In one embodiment a pharmaceutical composition includes the selected peptide(s) of SEQ ID NO: 10-18 or a related peptide as described herein. In another embodiment, the pharmaceutical composition employs mixtures of different peptides (e.g., mixtures of 2 through greater than 9, as described above for the diagnostic reagents). In another embodiment a pharmaceutical composition includes one or more nucleic acid sequences encoding one or more selected peptide(s) of SEQ ID NO: 10-18 or a related peptide as described herein. In another embodiment, the pharmaceutical composition employs mixtures of nucleic acid sequences encoding different peptides. Each pharmaceutical composition comprises the active component (peptide and/or nucleic acid) in an effective amount to induce an immune response and/or reduce the spread of infection and/or prevent an initial or repeated infection. In other embodiments, each composition includes a pharmaceutically acceptable carrier, excipient, or adjuvant.

By “pharmaceutically acceptable carrier or excipient” is meant a solid and/or liquid carrier, in in dry or liquid form and pharmaceutically acceptable. The compositions are typically sterile solutions or suspensions. Examples of excipients which may be combined with the antagonist or inhibitor include, without limitation, solid carriers, liquid carriers, adjuvants, amino acids (glycine, glutamine, asparagine, arginine, lysine), antioxidants (ascorbic acid, sodium sulfite or sodium hydrogen-sulfite), binders (gum tragacanth, acacia, starch, gelatin, polyglycolic acid, polylactic acid, poly-d,1-lactide/glycolide, polyoxacthylene, polyoxapropylene, polyacrylamides, polymaleic acid, polymaleic esters, polymaleic amides, polyacrylic acid, polyacrylic esters, polyvinylalcohols, polyvinylesters, polyvinylethers, polyvinylimidazole, polyvinylpyrrolidon, or chitosan), buffers (borate, bicarbonate, Tris-HCl, citrates, phosphates or other organic acids), bulking agents (mannitol or glycine), carbohydrates (such as glucose, mannose, or dextrins), clarifiers, coatings (gelatin, wax, shellac, sugar or other biological degradable polymers), coloring agents, complexing agents (caffeine, polyvinylpyrrolidone, β-cyclodextrin or hydroxypropyl-β-cyclodextrin), compression aids, diluents, disintegrants, dyes, emulsifiers, emollients, encapsulating materials, fillers, flavoring agents (peppermint or oil of wintergreen or fruit flavor), glidants, granulating agents, lubricants, metal chelators (ethylenediamine tetraacetic acid (EDTA)), osmo-regulators, pH adjustors, preservatives (benzalkonium chloride, benzoic acid, salicylic acid, thimerosal, phenethyl alcohol, methylparaben, propylparaben, chlorhexidine, sorbic acid, hydrogen peroxide, chlorobutanol, phenol or thimerosal), solubilizers, sorbents, stabilizers, sterilizer, suspending agent, sweeteners (mannitol, sorbitol, sucrose, glucose, mannose, dextrins, lactose or aspartame), surfactants, syrup, thickening agents, tonicity enhancing agents (sodium or potassium chloride) or viscosity regulators. See, the excipients in “Handbook of Pharmaceutical Excipients”, 5^{th} Edition, Eds.: Rowe, Sheskey, and Owen, APhA Publications (Washington, DC), 2005 and U.S. Pat. No. 7,078,053, which are incorporated herein by reference. The selection of the excipient is dependent on the nature of the compound selected and the form of administration desired.

Solid carriers include, without limitation, starch, lactose, dicalcium phosphate, microcrystalline cellulose, sucrose and kaolin, calcium carbonate, sodium carbonate, bicarbonate, lactose, calcium phosphate, gelatin, magnesium stearate, stearic acid, or talc. Fluid carriers without limitation, water, e.g., sterile water, Ringer's solution, isotonic sodium chloride solution, neutral buffered saline, saline mixed with serum albumin, organic solvents (such as ethanol, glycerol, propylene glycol, liquid polyethylene glycol, dimethylsulfoxide (DMSO)), oils (vegetable oils such as fractionated coconut oil, arachis oil, corn oil, peanut oil, and sesame oil; oily esters such as ethyl oleate and isopropyl myristate; and any bland fixed oil including synthetic mono- or diglycerides), fats, fatty acids (include, without limitation, oleic acid find use in the preparation of injectables), cellulose derivatives such as sodium carboxymethyl cellulose, and/or surfactants.

Examples of pharmaceutically acceptable adjuvants are well known in the art, sec, e.g. REMINGTON′S PHARMACEUTICAL SCIENCES, 18th Ed. (1990, Mack Publishing Co., Easton, Pa.) and GOODMAN AND GILMAN'S, THE PHARMACOLOGICAL BASIS OF THERAPEUTICS (10th ed. 2001). In certain embodiments, suitable adjuvants include the Adda Vax™ adjuvant used in the examples or Aluminum salts, such as aluminum hydroxide, aluminum phosphate, and aluminum potassium sulfate, Monophosphoryl lipid A, tetanus toxoid, cholera toxin B subunit, adenylate cyclase mutant, pertussis toxin mutant, and lipopolysaccharide, among other known adjuvants.

Thus, the pharmaceutical compositions may be in any form, i.e., a liquid, emulsion, dried powder, including as a lyophilized powder, and/or in a mist. In certain embodiments, the composition vaccine is lyophilized.

By “effective amount” is meant the amount or concentration (by single dose or in a dosage regimen delivered per day) of the peptide or nucleic acid composition sufficient to retard, suppress or inhibit B. burgdorferi infection, while providing the least negative side effects to the treated subject. In one embodiment, the effective amount of the peptide composition is within the range of 1 mg/kg body weight to 100 mg/kg body weight in humans including all integers or fractional amounts within the range. In certain embodiments, the effective amount is at least 2, 5, 10, 15, 20, 25, 30, 35, 40, 45, 50, 55, 60, 65, 70, 75, 80, 85, 90, 95 or 100 mg/kg body weight, including all integers or fractional amounts within the range. In one embodiment, the above amounts represent a single dose. In another embodiment, the above amounts define an amount delivered to the subject per day. In another embodiment, the above amounts define an amount delivered to the subject per day in multiple doses. In still other embodiments, these amounts represent the amount delivered to the subject over more than a single day. The nucleic acid compositions can be delivered in appropriate doses depending upon whether they are present as viral vectors, plasmid vectors and/or “naked DNA”. Selection of such dosages is within the skill of the art.

Therefore, in another aspect, a method for treating, inhibiting, or preventing Lyme Disease involves administering an effective amount of one of pharmaceutical compositions described herein to a subject. The administration is at a dosage or combination of dosages forming an amount effective to prevent or treat Lyme Disease. In another embodiment, the peptide(s) pharmaceutical composition or nucleic acid sequence(s) pharmaceutical composition described herein can also administered in combination with another an antibiotic or other pharmaceutical component designed to treat other aspects of the Lyme Disease infection.

Conventional and pharmaceutically acceptable routes of administration include, but are not limited to, systemic routes, such as intraperitoneal, intravenous, intranasal, intravenous, intramuscular, intratracheal, subcutaneous, and other parenteral routes of administration. In one embodiment, the route of administration is oral. In another embodiment, the route of administration is intraperitoneal. In another embodiment, the route of administration is intravascular. Routes of administration may be combined, if desired. In some embodiments, the administration is repeated periodically.

### Vaccine Sonicate and Methods of Use

In addition to the peptide/nucleic acid compositions described above, another a pharmaceutical composition for the treatment, inhibition, or prophylaxis of Lyme borreliosis comprises a sonicate of a Borrelia strain. In one embodiment, the sonicate is present in an amount sufficient to elicit an antibody independent protective immune response.

In one embodiment, a Borrelia sonicate is obtained by growing Borrelia burgdorferi, a selected strain or another Lyme Disease causing Borrelia species in a cell or cell line to produce the source B. burgdorferi for sonication, isolating the bacteria, and subjecting the isolated Borrelia to sonication. In certain aspects the present invention includes a composition that comprises an inactive Borrelia sonicate. The immunogenic compositions and/or vaccines of the present invention can further include an adjuvant or other excipient, as above described. See, e.g., similar methods disclosed in US Published Patent Application No. 2016/010625, incorporated by reference herein.

In general, after isolation bacteria are subjected to ultrasound treatment in liquid medium, at a frequency and intensity and for such duration as to rupture essentially all the bacteria, without at the same time raising the temperature of the bacterial solution sufficiently to significantly denature components of the sonicated lysate. In certain aspects treatment in the liquid medium with sound waves at an appropriate frequency and of sufficiently high-power level produces cavitation, whereby the structure of the bacteria in the liquid is disrupted and dispersed. Cavitation produces violent pressure changes in the sonicated liquid at multiple microscopically spaced volume elements within the liquid. These pressure changes, which may be thousands of atmospheres in magnitude, break up any clusters of cells as well as disintegrate the cells themselves, if the cavitation is sufficiently intense, and shear their genetic material, deoxyribonucleic acid (DNA), or ribonucleic acid (RNA). The destructive force of the cavitation depends upon the surface tension of the sonicated liquid and the vapor pressure as well as the magnitude of the change in bubble size, which in turn depends upon the sound intensity and wave length. Other considerations include the effect of dissolved gases in the liquid and the control of temperature during the procedure.

In one embodiment, the frequency, intensity, and duration of the sonication is to disintegrate the bacteria cells without raising the temperature of the liquid. In addition, the DNA or RNA of the bacteria which encodes their genetic information is sheared and is no longer capable of directing cellular replication. The completeness of the disruption of the cells can be determined by known methods including the use of microscopic examination and attempted growth of bacteria from the sonicated preparation.

In one embodiment, the sonic waves are introduced into the liquid medium at a frequency of about 20 to about 40 kHz. The minimum intensity (power) of the sonic waves should be about 1 watt/cm² when utilizing a 20 kHz frequency. At this minimum level of power, cavitation is initiated. In one embodiment, the intensity level at about 20 kHz is 50 to 175 watts/cm². The destructive power of the sonic waves varies as a function of the frequency of the sonic waves used, with a lower effect being produced at higher frequencies.

In certain embodiments, the bacteria samples can be pulsed for about 15 seconds at about setting 4-5 (about 25% of output of sonicator, available from Rx Technologies, Inc., Garden City, N.Y., U.S.A.) and then rested for about 60 seconds in an ice bath. The sample is further sonicated for an additional 3 pulses (about 15 seconds). In general, the duration of sonication may range from about 10 seconds to several minutes or longer and most preferably is performed in at least two installments for about 15 seconds or longer. The sonication duration and intensity schedule are provided which results in maximum lysis and removes any viable bacteria from the sonicate.

After sonication of the bacteria the resulting product (a sonicate) is directly available for use as a pharmaceutical composition or vaccine. No further additions or purification of the resulting material is necessary. Alternatively, the sonicated material can be added to a pharmaceutically acceptable carrier or adjuvant. Suitable pharmaceutically acceptable carriers will be apparent to those skilled in the art, as described.

Thus, in one aspect a method of treating, preventing, or retarding infection by B. burgdorferi comprises administering an effective amount of the B. burgdorferi sonicate to a subject in need thereof, wherein said sonicate elicits an antibody-independent protective immune response. The sonicate vaccines may be administered as a liquid, emulsion, dried powder, including as a lyophilized powder, and/or in a mist through any suitable parenteral route, intravenously, intraperitoneally, intradermally, by scarification, subcutaneously, intramuscularly, or inoculated by a mucosal route, e.g., orally, intranasally, as an aerosol, by eye drop, or implanted as a freeze-dried powder.

Dosages of the Borrelia (e.g., B. burgdorferi) associated with adjuvants and/or carriers will often be about that of the immunogenic material (disrupted bacteria) alone. For use as human vaccines, dosages will be set by the prescribing physician considering relevant factors including the age, weight and condition of the patient and the pharmacokinetics of the agent and release characteristics of the agent from pharmaceutical dosage forms. In one embodiment the vaccine will generally contain an amount of protein ranging from about 25 micrograms to about 5 milligrams. Thus, the amount of protein used will fall within this range. The amount of protein contained in any sonicate can be generally assayed by the methods of Bradford, Anal. Biochem., 72,248 (1976) or Lowry, J. Biol. Chem., 193, 265 (1951).

### Lyme Disease

Lyme Disease is caused by Borrelia burgdorferi (or other Borrelia species) transmitted by ticks. Human infection can result in musculoskeletal, neurologic or cardiovascular disorders, which are normally present in three phases: (1) early localized disease; (2) early disseminated disease; and (3) late Lyme disease. The clinical features of each phase can overlap, and some patients present in later phases of the disease without previously having symptoms of earlier phases of the disease. Early localized disease generally states within two weeks of the tick bite and often starts with a characteristic skin rash called erythema migrans (EM). The disease progresses to the second early disseminated state where the EM rash is followed by Spirochetemia caused by early wide-spread dissemination of the bacteria through tissue and body fluids. This second stage may be accompanied by one or more additional skin lesions, fatigue, myalgia, arthralgia, neurologic and cardiac symptoms. Left untreated, the disease progresses to late Lyme disease where chronic major manifestations occur. Late stage Lyme disease is characterized by arthritis, encephalomyelitis and/or peripheral neuropathy, and potentially other symptoms. Approximately 25,000 to 30,000 cases of Lyme disease are reported in the United States annually, with CDC estimates of actual incidence reaching ten times that amount in view of underreporting; additional cases have also been reported in Europe and Asia.

In certain embodiments, the compositions and methods described herein are capable of detecting and/or treating Lyme disease in the early localized disease phase, the early disseminated disease phase, or the late Lyme disease phase. In certain embodiments, the patient is symptomatic. In certain embodiments, the patient is asymptomatic. In certain embodiments the patient does not have erythema migrans before or after being diagnosed.

Clinical diagnosis of Lyme disease is usually based on a typical EM rash in the early stage of the disease, and treatment of the disease with oral antibiotics is generally effective at the early stage. However, the EM rash can be either missed (e.g., it usually disappears in a few days) or not present in an infected person (e.g., occurs in 70-80% of infected people). Without a confirmed EM rash, a clear diagnosis of Lyme disease can be difficult for a number of reasons, one of which is lack of specific signs and symptoms. For example, Lyme disease may mimic other conditions, such as chronic fatigue, multiple sclerosis, rheumatoid arthritis, and other diseases with multiple symptoms involving different body systems. In certain embodiments, the compositions and methods described herein are capable of treating complications and symptoms caused by Lyme disease. In certain embodiments, the compositions and methods described herein are capable of halting the progression of Lyme disease prior to the patient developing serious complications or chronic symptoms.

Arthritis is a prominent manifestation in patients with Lyme disease. The majority of individuals with Lyme disease have the HLA-DRB1*0401 or HLA-DRB1*0101 allele; these alleles also occur more frequently in patients with rheumatoid arthritis. HLA-DRB1 belongs to MHC II and is recognized by T cell. We have selected the epitope regions of the antigenic proteins that bind to HLA-DRB1.

### EXAMPLES

The following examples disclose specific embodiments of diagnostic and prophylactic reagents for the diagnosis of and prevention or treatment of Borrelia infection.

### Example 1: Identification of B Cell Epitope Peptides of Borrelia

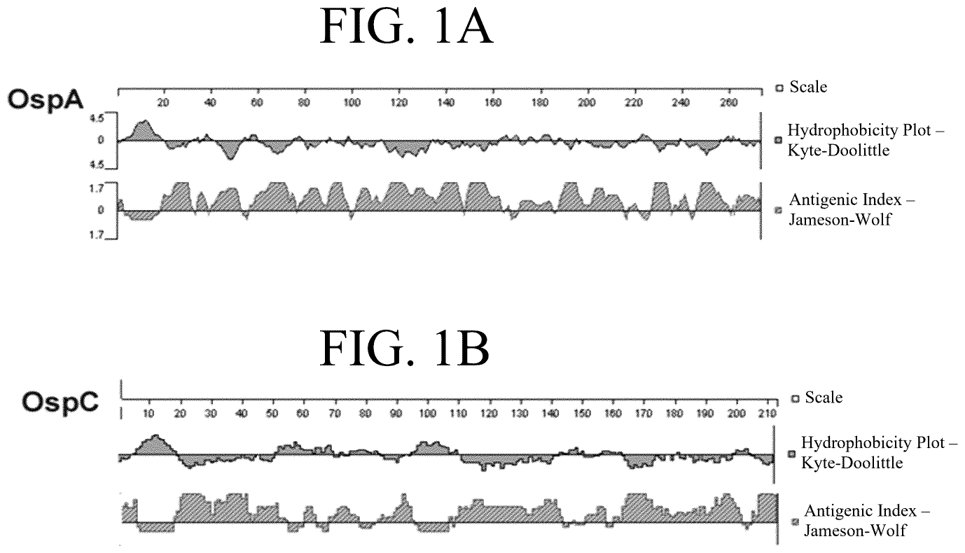

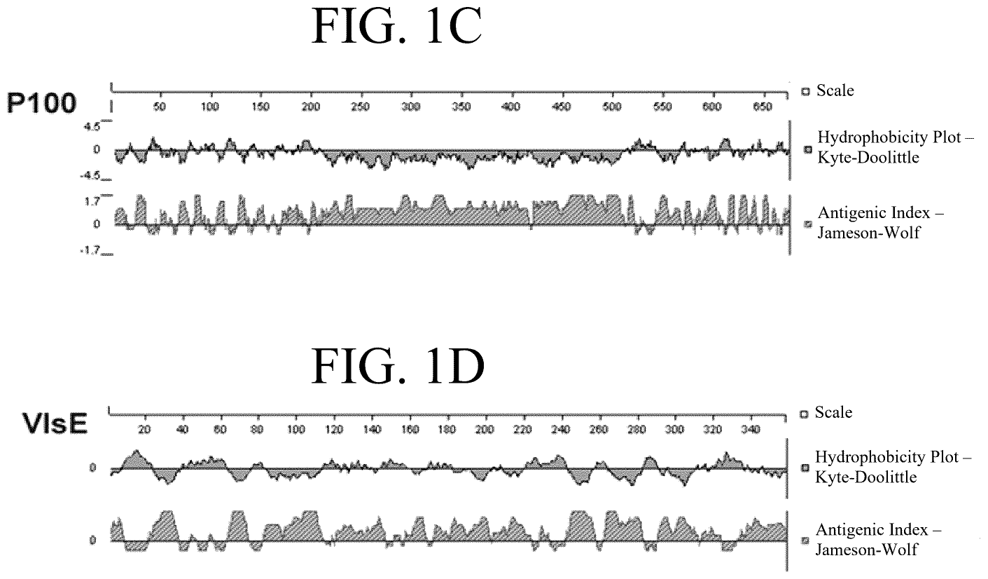

We determined the epitopes of the antigenic proteins of Borrelia OspA, OspC, OspE, OspF, VISE, BmpA, napA, p41, and p100 using a bioinformatice approach (FIGS. 1A-1D and Table 1). To determine a protein sequence for potential antigenic epitopes, sequences that are hydrophilic, surface-oriented, and flexible are selected. Most naturally occurring proteins in aqueous solutions have their hydrophilic residues on the protein surface and hydrophobic residues buried in the interior. We selected protein sequences that had good hydrophilicity as predicted by the Lasergene software (DNAStar, WI, USA). Highlighted sequences demonstrate peptides for use as diagnostic, treatment or vaccine compositions.

TABLE 1
Outer Surface Protein Sequences of
B. burgdorf antigenic proteins

| | | SEQ |
|---|---|---|
| | | ID |
| Protein | Amino Acid Sequence | NO |
| OspA | MKKYLLGIGL ILALIACKQN VSSLDEKNSV SVDLPGEMKV | 1 |
| | LVSKEKNKDG KYDLIATVDK LELKGTSDKN NGSGVLEGVK | |
| | ADKSKVKLTI SDDLGQTTLE VFKEDGKTLV SKKVTSKDKS | |
| | STEEKFNEKG EVSEKIITRA DGTRLEYTGI KSDGSGKAKE | |
| | VLKGYVLEGT LTAEKTTLVV KEGTVTLSKN ISKSGEVSVE | |
| | LNDTDSSAAT KKTAAWNSGT STLTITVNSK KTKDLVFTKE | |
| | NTITVQQYDS NGTKLEGSAV EITKLDEIKN ALK | |
| OspC | MKKNTLSAIL MTLFLFISCN NSGKDGNTSA NSADESVKGP | 2 |
| | NLTEISKKIT ESNAVVLAVK EVETLLASID EVAKKAIGNL | |
| | IAQNGLNAGA NQNGSLLAGA YVISTLIAEK LDGLKNSEEL | |
| | KEKIEDAKKC NKAFTDKLKS SHAELGIANG AATDANAKAA | |
| | ILKTNGTKDK GAQELEKLFE SVKNLSKAAQ ETLNNSVKEL | |
| | TSPVVAESPK KP | |
| OspE | MNKKMKMFIV YAVFILIGAC KIHTSYDEQS SGESKVKKIE | 3 |
| | FSKFTVKIKN KDKSGNWTDL GDLVVRKEEN GIDTGLNAGG | |
| | HSATFFSLEE EVVNNFVKVM TEGGSFKTSL YYGYKEEQSV | |
| | INGIQNKEII TKIEKIDGTE YITFSGDKIK NSGDKVAEYA | |
| | ISLEELKKNL K (171) | |
| OspF | MNKKMFIICA VFALIISCKN YATSKDLEGA VQDLESSEQN | 4 |
| | VKKTEQEIKK QVEGFLEILE TKDLNKLDTK EIEKRIQELK | |
| | EKIEKLDSKK TSIETYSEYE EKLKQIKEKL KGKADLEDKL | |
| | KGLEDSLKKK KEERKKALED AKKKFEEFKG QVGSATGVTT | |
| | GHRAGNQGSI GAQAWQCANS LGLGVSYSSS TGTDSNELAN | |
| | KVIDDSIKKI DEELKNTIEN NGEVKKE | |
| OSP | MNTKKISSAI LLTTFFVFIN CKSQVADKDD PTNKFYQSVI | 5 |
| VlsE | QLGNGFLDVF TSFGGLVAEA FGFKSDPKKS DVKTYFTTVA | |
| | AKLEKTKTDL NSLPKEKSDI SSTTGKPDST GSVGTAVEGA | |
| | IKEVSELLDK LVKAVKTAEG ASSGTAAIGE VVDNAAAAKA | |
| | ADKDSVTGIA KGIKEIVEAA GGSKKLKAAA AKGENNKGAG | |
| | KLFGKAGDAA HGDSEAASKA AGAVSAVSGE QILSAIVKAA | |
| | AAGDQEGKKP GEAKNPIAAA IGEGDGDAEF NQDGMKKDDQ | |
| | IAAAIALRGM AKDGKFAVKN DEKGKAEGAI KGAAESAVRK | |
| | VLGAITGLIG DAVSSGLRKV GDSVKAASKE TPPALNK | |
| NapA | MEKYLSYIKK DDLDAIQLKL QELLASLHIF YSNLRGIHWN | 6 |
| | IKDTNFFVIH KKTQKLYEYI EKIIDIVAER SRMLGYDSEF | |
| | RYSEFMKKSF IKELDIESTS NFLPSMESIV CSLTEILKNI | |
| | FGMRKLIDTA GDYGTANIMD DIMSDLEKHL WMHKALLENC | |
| | DCFCHDENES KCCECDAK | |
| P41 | MIINHNTSAI NASRNNAINA ANLSKTQEKP SSGYRINRAS | 7 |
| | DDAAGMGVSG KINAQIRGLS QASRNTSKAI NFIQTTEGNL | |
| | NEVEKVLVRM KELAVQSGNG TYSDSDRGSI QIEIEQLTDE | |
| | INRIADQAQY NQMHMLSNKS ASQNVKTAEE LGMQPAKINT | |
| | PASLSGSQAS WTLRVHVGAN QDEAIAVNIY SANVANLFAG | |
| | EGAQAAQAAP VQEGAQEEGA QQPTPATAPS QGGVNSPVNV | |
| | TTTVDANTSL AKIENAIRMI SDQRANLGAF QNRLESIKNS | |
| | TEYAIENLKA SYAQIKDATM TDEVVAATTK SILTQSAMAM | |
| | IAQANQVPQY VLSLLR | |
| P100 | MKELDKEKLR DFVNMDLEFV NYKGPYDSTN TYEQIVGIGE | 8 |
| | FLARPLINSN SNSIYYGKYF INRFIDDQDK KASVDVFSIG | |
| | SRSQLDSILN LRRILTGYLI KSFDYERSSA ELIAKVITIH | |
| | NAVYRGDLNY YKEVYIEAAL KSLTKENAGL SRVYSQWAGK | |
| | TQIFIPLKKN ILSGKVESDI DIDSLVTDKV VAALLSENEA | |
| | GVNFARDITD IQGETHKADQ DKIDIELDNV HKSDSNITET | |
| | IENLRDQLEK ATDEEHRKEI ESQVDAKKKQ KEELDKKAID | |
| | LDKAQQKLDS SEDNLDIQRD TVREKIQEDI DEINKEKNLP | |
| | KPGDVSSPKV DKQLQIKESL EDLQEQLKET SDENQKREIE | |
| | KQIEIKKSDE ELLKSKDPKA LDLNGDLNSK VSSKEKIKGK | |
| | EGEIVKEESK ASLADLNNDE NLMRPEDQKL SEDKKLDSKK | |
| | NLKPVSEIER VNEISKSNNN EISESSPLYK PSYSDMDSKE | |
| | GIDNKDVNLQ ETKSQTKSQP TSLNQDLTTM SIDSSNPVFL | |
| | EVIDPITNLG TLQLIDLNTG VRLKESTQQG IQRYGIYERE | |
| | KDLVVIKMDS GKAKLQILNK LENLKVISES NFEINKNSSL | |
| | YVDSKMILVV VKDSGNVWRL AKFSPKNLNE FILSENKILP | |
| | FTSFSVRKNF IYLQDEFKSL ITLDLNTLKK VK | |
| BmpA | MNKILLLILL ESIVFLSCSG KGSLGSEIPK VSLIIDGTFD | 9 |
| | DKSFNESALN GVKKVKEEFK IELVLKESSS NSYLSDLEGL | |
| | KDAGSDLIWL IGYRFSDVAK VAALQNPDMK YAIIDPIYSN | |
| | DPIPANLVGM TFRAQEGAFL TGYIAARLSK TGKIGFLGGI | |
| | EGEIVDAFRY GYEAGAKYAN KDIKIFTQYI GSFADLEAGR | |
| | SVATRMYSDE IDIIHHAAGL GGIGAIEVAK ELGSGHYIIG | |
| | VDEDQAYLAP DNVITSTTKD VGRALNIFTS NHLKTNTFEG | |
| | GKLINYGLKE GVVGFVRNPK MISFELEKEI DNLSSKIINK | |
| | EIIVPSNKES YEKFLKEFI | |

### Example 2: Lyme Disease Detection-ELISA Assay

The following peptides identified in Table 2 were selected as epitopes of the N-terminal B. burgdorferi proteins of Table 1 for peptide synthesis. We synthesized the following peptides (GenScript) reported in Table 2:

TABLE 2
Peptides from Borrelia burgdorferi

| Derived from | | |
|---|---|---|
| Protein | Amino Acid Sequence | SEQ ID NO |
| OspA | KVTSKDKSSTEEKFNEKGEVS | 10 |
| OspC | NSGKDGNTSANSADESVKGP | 11 |
| OspE | FILIGACKIHTSYDEQSSGESK | 12 |
| OspF | VQDLESSEQNVKKTEQEIKK | 13 |
| VlsE | FFVFINCKSQVADKDDPTNKFY | 14 |
| BmpA | DKSFNESALNGVKKVKEEFK | 15 |
| napA | SYIKKDDLDAIQLKLQELLASL | 16 |
| p41 | LSKTQEKLSSGYRINRASDDA | 17 |
| p100 | YKGPYDSTNTYEQIVGIGEFLAR | 18 |

To determine whether the antigenic peptides of Borrelia could be used in diagnostics we probed the sera of patients infected with Borrelia.

Previously we have observed that for diagnostic applications multiple peptides are better probes than single peptides. Hence, equal amounts of all the peptides of Table 2 were diluted (5 micrograms/ml in PBS) and coated on an ELISA plate (Nunc, MaxiSorp, ThermoFisher). The plates were incubated for 48 hours at 4° C. After washing, they were incubated with the sera of patients infected with Borrelia or healthy controls. These samples were further probed with anti-human horseradish peroxidase (HRP) and the data recorded after the addition of substrate.

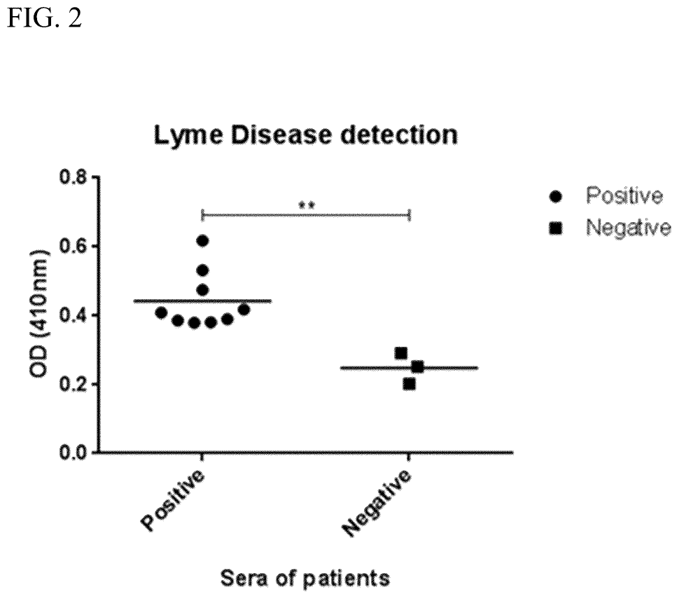

From the results demonstrated in FIG. 2, the Lyme disease patient samples had higher values compared to the healthy controls. These ELISA data demonstrated that the peptides of Table 2 are useful in detecting anti-B. burgdorferi antibodies in infected subjects. Thus, these peptides are useful in a variety of diagnostic methods to detect Lyme Disease.

### Example 3: Sonix Vaccine

In addition to peptide immunogens, we also used Borrelia burgdorferi to create a sonicated material useful as an immunogen (referred to as the “Sonix” vaccine). Previously, we demonstrated that sonicated Ehrlichia are antibody independent highly potent vaccine candidates. See, e.g., US Patent Publication No. 2016/0106825, incorporated by reference herein. The procedures for Ehrlichia as described were substantially used to create the Borrelia Sonix vaccine.

To create this vaccine composition, sonic waves were introduced into the liquid medium (e.g., PBS buffer) of a Borrelia bacterial sample at a frequency of about 20 to about 40 kHz. The minimum intensity (power) of the sonic waves was about 1 watt/cm² when utilizing a 20 kHz frequency. At this minimum level of power, cavitation is initiated. Preferably, the intensity level at about 20 kHz was 50 to 175 watts/cm². In the case of Borrelia, the cavitation intensity and frequency was 20 kHz ranging from about 20 to about 150 watts/cm², or about 60-100 watts/cm². The samples were pulsed for about 30 seconds at the “hold” setting (about 25% of output of sonicator) and then rested for about 60 seconds in an ice bath. The sample was further sonicated for an additional 3 pulses (about 30 seconds). The duration of sonication may be several minutes or longer and most preferably is performed in at least three installments for about 30 seconds or longer. The sonication duration and intensity schedule resulted in maximum lysis and removal of any viable bacteria from the sonicate.

In certain aspects the sonication was conducted at room pressure and the sample maintained at a temperature of roughly about zero to 5° C. to avoid heating and to reduce the activity of cellular enzymes. After sonication of the bacteria the resulting product (Sonix vaccine) was directly available for use as a vaccine. No further additions or purification of the resulting material was necessary. The sonicated vaccine is suitable for injection as is, or for convenience of administration can be added to a pharmaceutically acceptable carrier or adjuvant.

### Example 4: Protection Against Challenge With Lyme Disease

(a) To determine whether the peptides of Table 2 could protect against Borrelia infection, we immunized C3H/HeN mice with 50 μg (each) of the peptides in the presence of the adjuvant (Adda Vax) (0.2 ml) (subcutaneous immunization). The mice were immunized with two doses of the peptides two weeks apart. Two weeks after the second immunization, they were challenged with 10⁵ Borrelia cells per mouse. Two weeks post-infection, mice were euthanized and tissues were collected to evaluate bacterial burden. Sera were isolated from the blood that was collected before and after bacterial challenge.
(b) We also sonicated Borrelia burgdorferi and used this material as an immunogen (referred to as the “Sonix” vaccine). We immunized C3H/HeN mice with about 50 micrograms of the Sonix vaccine in the presence of the adjuvant (AddaVax™) (0.2 ml) subcutaneously. An optional second dose is administered two weeks later. Two weeks after the second immunization, they were challenged with 10⁵ Borrelia cells per mouse. Two weeks post-infection, mice were euthanized and tissues were collected to evaluate bacterial burden. Sera were isolated from the blood that was collected before and after bacterial challenge.
(c) Antibody Induction:

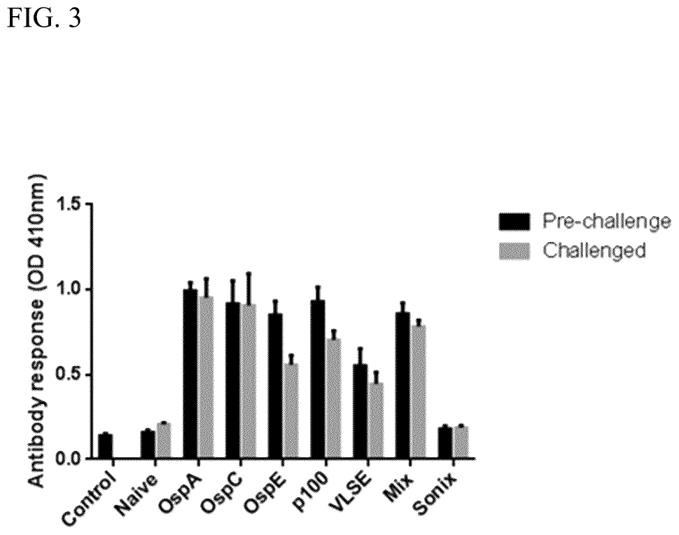

As illustrated in the results of FIG. 3, the peptides of the antigenic proteins of Borrelia reactivated with peptide-specific antibody before and after challenge with the pathogen. However, there was a decrease in antibody response after challenge irrespective of the peptides used. The Sonix vaccine did not induce any antibody after immunization or after challenge with the pathogen.
(d) Bacterial Load in Tissues After Vaccination

Borrelia is localized in the joints and heart tissues. The mouse tissues were collected after euthanization on day 14 after challenge and subjected to qPCR.

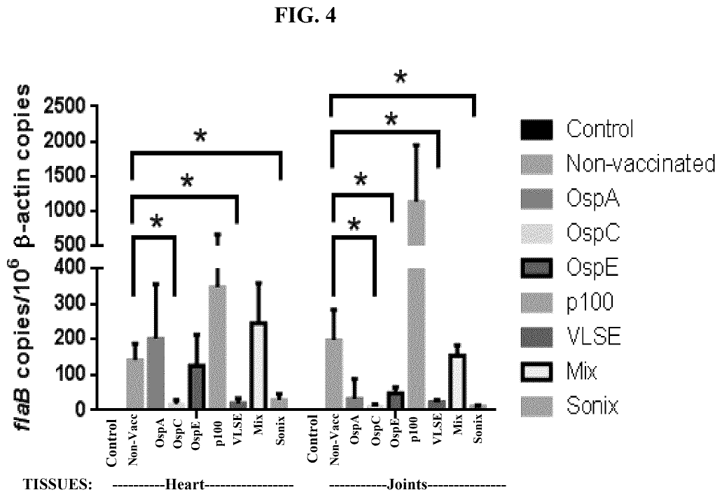

As demonstrated in the graph of FIG. 4, the naïve mice (unvaccinated) had the highest bacterial load in the heart and joint tissues. Mice immunized with OspC and VIsE had a significant reduction in the bacterial load in the heart and joint, demonstrating that the peptides provided protection against Borrelia.

Mice immunized with OspE had significantly lower levels of bacterial burden in the joint compared to the heart.

However, the peptide of p100 used in this test provided no significant protection against Borrelia.

The Sonix vaccine also provided protection against Borrelia. The heart and joint tissues had low copy number of the bacterial pathogen demonstrating that the Sonix vaccine is a highly potent vaccine candidate for Lyme disease.

These examples demonstrate that that compositions containing the identified B. burgdorferi peptides as well as the bacterial sonicate (Sonix) provide significant protection against the pathogenic Borrelia and are useful in vaccine compositions for Lyme disease (FIG. 4).

Each and every patent, patent application, and publication, including websites cited throughout specification are incorporated herein by reference. Similarly, the SEQ ID NOs which are referenced herein and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

### EMBODIMENTS

1. A reagent useful for the diagnosis of Lyme borreliosis in a subject comprising:
   (a) a peptide of SEQ ID NO: 10;
   (b) a peptide of SEQ ID NO: 11;
   (c) a peptide of SEQ ID NO: 12;
   (d) a peptide of SEQ ID NO: 13;
   (e) a peptide of SEQ ID NO: 14;
   (f) a peptide of SEQ ID NO: 15;
   (g) a peptide of SEQ ID NO: 16;
   (h) a peptide of SEQ ID NO: 17;
   (i) a peptide of SEQ ID NO: 18
   (j) a peptide having at least 90% identity to a peptide of any of SEQ ID NO: 10 to 18;
   (k) a peptide variant differing in one or more conservative amino acids to a peptide of any of SEQ ID NO: 10-18; or
   (l) a peptide variant differing in one or more naturally occurring amino acids from any of a peptide of SEQ ID NO: 10-18 based upon the strain of B. burgdorferi from which the peptide sequence is derived.
2. The reagent of embodiment 1, comprising a mixture of two or more peptides (a) through (l).
3. The reagent of embodiment 2, wherein said mixture comprises a combination of at least 3, 4, 5, 6, 7, 8, 9, 10 of said peptides (a)-(l).
4. The reaction of embodiment 1, comprising a combination of 2, 3, 4, 5, 6, 7, 8, or 9 of the peptides (a) through (i).
5. A reagent useful for the diagnosis of Lyme borreliosis in a subject comprising a nucleic acid sequence encoding:
   (a) a peptide of SEQ ID NO: 10;
   (b) a peptide of SEQ ID NO: 11;
   (c) a peptide of SEQ ID NO: 12;
   (d) a peptide of SEQ ID NO: 13;
   (e) a peptide of SEQ ID NO: 14;
   (f) a peptide of SEQ ID NO: 15;
   (g) a peptide of SEQ ID NO: 16;
   (h) a peptide of SEQ ID NO: 17;
   (i) a peptide of SEQ ID NO: 18
   (j) a peptide having at least 90% identity to a peptide of any of SEQ ID NO: 10 to 18;
   (k) a peptide variant differing in one or more conservative amino acids to a peptide of any of SEQ ID NO: 10-18; or
   (l) a peptide variant differing in one or more naturally occurring amino acids from any of a peptide of SEQ ID NO: 10-18 based upon the strain of B. burgdorferi from which the peptide sequence is derived.
6. The reagent of embodiment 5, comprising a mixture of nucleic acid sequences, each nucleic acid sequence encoding a different peptide (a) through (l).
7. The reagent of embodiment 6, wherein said mixture comprises at least 3, 4, 5, 6, 7, 8, 9, 10 of said nucleic acid sequences, each nucleic acid sequence encoding a different peptide (a) through (l).
8. The reagent of any of embodiments 1 or 4, wherein one or more of said peptides (a) to (l) or nucleic acid sequences encoding same is immobilized on an immobilization surface.
9. The reagent of any of embodiments 1 or 4, wherein one or more of said peptides or nucleic acid molecules is covalently or noncovalently joined to a molecule or moiety capable alone or in combination with one or more additional molecules of generating a detectable signal.
10. A method of diagnosing Lyme borreliosis in a subject, comprising contacting in vitro a biological sample from the subject in with a reagent of embodiments 1 or 4; and detecting in said sample the presence of an antibody that binds one or more of said peptides.
11. The method of embodiment 10, wherein the biological sample is whole blood, plasma, serum, ascites fluid, peritoneal fluid and a tissue or fluid from a tissue or joint sample.
12. A pharmaceutical composition for the treatment, retardation or prophylaxis of Lyme borreliosis comprising:
   (a) a peptide of SEQ ID NO: 10;
   (b) a peptide of SEQ ID NO: 11;
   (c) a peptide of SEQ ID NO: 12;
   (d) a peptide of SEQ ID NO: 13;
   (e) a peptide of SEQ ID NO: 14;
   (f) a peptide of SEQ ID NO: 15;
   (g) a peptide of SEQ ID NO: 16;
   (h) a peptide of SEQ ID NO: 17;
   (i) a peptide of SEQ ID NO: 18
   (j) a peptide having at least 90% identity to a peptide of any of SEQ ID NO: 10 to 18;
   (k) a peptide variant differing in one or more conservative amino acids to a peptide of any of SEQ ID NO: 10-18; or
   (l) a peptide variant differing in one or more naturally occurring amino acids from any of a peptide of SEQ ID NO: 10-18 based upon the strain of B. burgdorferi from which the peptide sequence is derived; or
   (m) a mixture of peptides selected from (a) through (l); and a pharmaceutically acceptable carrier, excipient or adjuvant.
13. The compositions of embodiment 12 comprising a mixture of peptides selected from (a) through (l).
14. A pharmaceutical composition for the treatment, retardation or prophylaxis of Lyme borreliosis comprising a nucleic acid sequence encoding
   (a) a peptide of SEQ ID NO: 10;
   (b) a peptide of SEQ ID NO: 11;
   (c) a peptide of SEQ ID NO: 12;
   (d) a peptide of SEQ ID NO: 13;
   (e) a peptide of SEQ ID NO: 14;
   (f) a peptide of SEQ ID NO: 15;
   (g) a peptide of SEQ ID NO: 16;
   (h) a peptide of SEQ ID NO: 17;
   (i) a peptide of SEQ ID NO: 18
   (j) a peptide having at least 90% identity to a peptide of any of SEQ ID NO: 10 to 18;
   (k) a peptide variant differing in one or more conservative amino acids to a peptide of any of SEQ ID NO: 10-18; or
   (l) a peptide variant differing in one or more naturally occurring amino acids from any of a peptide of SEQ ID NO: 10-18 based upon the strain of B. burgdorferi from which the peptide sequence is derived; or
   (m) a mixture of peptides selected from (a) through (l); and a pharmaceutically acceptable carrier, excipient or adjuvant.
15. The composition of embodiment 14 comprising a mixture of nucleic acid sequences, each nucleic acid sequence encoding a different peptide (a) through (l).
16. A pharmaceutical composition for the treatment, retardation or prophylaxis of Lyme borreliosis comprising a sonicate of Borrelia burgdorferi.
17. The composition of embodiment 16, wherein the sonicate is present in an amount sufficient to elicit an antibody independent protective immune response.
18. A method of treating, preventing or retarding infection by comprising administering an effective amount of the pharmaceutical composition of embodiment 16 to a subject in need thereof, wherein said sonicate elicits an antibody-independent protective immune response.

1. A reagent useful for the diagnosis of Lyme borreliosis in a subject comprising a polynucleotide encoding a fusion protein comprising:
i) at least one outer surface protein consisting of:
a) a peptide of SEQ ID NO: 10;
(b) a peptide of SEQ ID NO: 11;
(c) a peptide of SEQ ID NO: 12;
(d) a peptide of SEQ ID NO: 13;
(e) a peptide of SEQ ID NO: 14;
(f) a peptide of SEQ ID NO: 15;
(g) a peptide of SEQ ID NO: 16;
(h) a peptide of SEQ ID NO: 17;
(i) a peptide of SEQ ID NO: 18
(j) a peptide having at least 90% identity to a peptide of any of SEQ ID NO: 10 to 18;
(k) a peptide variant differing in one or more conservative amino acids to a peptide of any of SEQ ID NO: 10-18; or
(l) a peptide variant differing in one or more naturally occurring amino acids from any of a peptide of SEQ ID NO: 10-18 based upon the strain of B. burgdorferi from which the peptide sequence is derived; and
ii) at least one additional outer surface protein of:
(m) a peptide of SEQ ID NO: 10;
(n) a peptide of SEQ ID NO: 11;
(o) a peptide of SEQ ID NO: 12;
(p) a peptide of SEQ ID NO: 13;
(l) a peptide of SEQ ID NO: 14;
(r) a peptide of SEQ ID NO: 15;
(s) a peptide of SEQ ID NO: 16;
(t) a peptide of SEQ ID NO: 17;
(u) a peptide of SEQ ID NO: 18;
(v) a peptide having at least 90% identity to a peptide of any of SEQ ID NO: 10 to 18; or
(w) a peptide variant differing in one or more conservative amino acids to a peptide of any of SEQ ID NO: 10-18,
wherein the at least one sequence of i) is not the same as the at least one sequence of ii).

2. The reagent of claim 1, wherein said fusion protein comprises a combination of at least 3, 4, 5, 6, 7, 8, 9, or 10 of said peptides (m)-(w).

3. The reagent of claim 1, wherein the polynucleotides encoding said peptides (a) to (w) are immobilized on an immobilization surface.

4. The reagent of claim 1, wherein said are covalently or noncovalently joined to a molecule or moiety capable alone or in combination with one or more additional molecules of generating a detectable signal.

5. A method of diagnosing Lyme borreliosis in a subject, comprising contacting in vitro a biological sample from the subject in with a reagent of claim 1; and
detecting in said sample the presence of an antibody that binds one or more of said peptides.

6. The method of claim 5, wherein the biological sample is whole blood, plasma, serum, ascites fluid, peritoneal fluid or a tissue or fluid from a tissue or joint sample.

7. A pharmaceutical composition for the treatment, retardation, or prophylaxis of Lyme borreliosis comprising a pharmaceutically acceptable carrier, an excipient or adjuvant, and the reagent of claim 1.

8. The composition of claim 7, wherein said fusion protein comprises a combination of at least 3, 4, 5, 6, 7, 8, 9, or 10 of said peptides (m)-(w).

9. The composition of claim 7, wherein said peptides are covalently or noncovalently joined to a molecule or moiety capable alone or in combination with one or more additional molecules of generating a detectable signal.

10. The reagent of claim 1, wherein said composition comprises a fusion protein of 4 of said peptides (m)-(w).

11. The composition of claim 7, wherein said composition comprises a fusion protein of 4 of said peptides (m)-(w).

12. A reagent useful for the diagnosis of Lyme borreliosis in a subject comprising a fusion protein having:
i) at least one outer surface protein consisting of:
a peptide of SEQ ID NO: 10;
(b) a peptide of SEQ ID NO: 11;
(c) a peptide of SEQ ID NO: 12;
(d) a peptide of SEQ ID NO: 13;
(e) a peptide of SEQ ID NO: 14;
(f) a peptide of SEQ ID NO: 15;
(g) a peptide of SEQ ID NO: 16;
(h) a peptide of SEQ ID NO: 17;
(i) a peptide of SEQ ID NO: 18
(j) a peptide having at least 90% identity to a peptide of any of SEQ ID NO: 10 to 18;
(k) a peptide variant differing in one or more conservative amino acids to a peptide of any of SEQ ID NO: 10-18; or
(l) a peptide variant differing in one or more naturally occurring amino acids from any of a peptide of SEQ ID NO: 10-18 based upon the strain of B. burgdorferi from which the peptide sequence is derived; and
ii) at least one additional outer surface protein of:
(m) a peptide of SEQ ID NO: 10;
(n) a peptide of SEQ ID NO: 11;
(o) a peptide of SEQ ID NO: 12;
(p) a peptide of SEQ ID NO: 13;
(l) a peptide of SEQ ID NO: 14;
(r) a peptide of SEQ ID NO: 15;
(s) a peptide of SEQ ID NO: 16;
(t) a peptide of SEQ ID NO: 17;
(u) a peptide of SEQ ID NO: 18;
(v) a peptide having at least 90% identity to a peptide of any of SEQ ID NO: 10 to 18; or
(w) a peptide variant differing in one or more conservative amino acids to a peptide of any of SEQ ID NO: 10-18,
wherein the at least one sequence of i) is not the same as the at least one sequence of ii).
